(19) United States
(12) Patent Application Publication
(10) Pub. No.: US 2026/0016054 A1
(43) Pub. Date: Jan. 15, 2026

(54) Rotary Driving Module and Medical Imaging Equipment

(71) Applicant: Shanghai United Imaging Healthcare Co., Ltd., Shanghai (CN)

(72) Inventors: Guoxuan Qin, Shanghai (CN); Chengzhe Zhang, Shanghai (CN); Guangbiao Chen, Shanghai (CN); Junwen Lin, Shanghai (CN)

(21) Appl. No.: 19/335,000

(22) Filed: Sep. 22, 2025

Related U.S. Application Data
(63) Continuation of application No. PCT/CN2024/082996, filed on Mar. 21, 2024.

(30) Foreign Application Priority Data
Mar. 21, 2023 (CN) 202310280138.0

(51) Int. Cl. F16D 63/00; A61B 6/00; F16D 121/22; F16H 37/02

(52) U.S. Cl. CPC F16D 63/00; A61B 6/4441; F16D 2121/22; F16H 37/02

(57) ABSTRACT

A rotary driving module and a medical imaging equipment, wherein the rotary driving module includes a torque transmission structure and a braking structure. The torque transmission structure includes a first transmission system and a second transmission system connected to an output shaft of a motor. The first transmission system and the second transmission system are connected. The braking structure is connected to the second transmission system and is configured to brake the second transmission system when the working state of the motor conforms to the preset condition.

### CROSS-REFERENCE TO RELATED APPLICATIONS

This application is a continuation of International Application No. PCT/CN2024/082996, filed on Mar. 21, 2024, which claims priority for a Chinese patent application No. 202310280138.0, filed on Mar. 21, 2023, titled “Rotary driving module and medical imaging equipment”, the entire contents of each of which are hereby incorporated by reference.

### TECHNICAL FIELD

This application relates to the technical field of medical devices, particularly to a rotary driving module and a medical imaging equipment.

### BACKGROUND

With the improvement of medical standards, Digital Subtraction Angiography (DSA) equipment has become increasingly common in hospitals, and DSA equipment is used in diagnostic and interventional surgeries in major hospitals. Typically, to meet the needs of clinical diagnosis, a C-arm that drives the DSA device is used as the driving component for rotational movement.

On the one hand, the shaft with the largest load in the DSA equipment, which is connected to the drive component to drive the rotation of the C-arm, has a large load requirement. The volume and weight of the reducer and motor that are matched with this shaft are large, which results in the overall weight of the drive component being relatively heavy, and thus the maintainability of the drive component is poor.

On the other hand, for the current DSA equipment's C-arm drive component, when the brake at the motor end has a single failure due to a large eccentric load, the C-arm is very likely to lose control and crush the doctor or patient, creating a safety hazard.

### SUMMARY

The present application provides a rotary driving module and a medical imaging equipment with the aim of avoiding the safety hazard caused by the loss of control of the C-arm when the brake of the motor fails.

To achieve this purpose, on the one hand, the application provides a rotary driving module, including: a torque transmission structure, wherein the torque transmission structure includes a first transmission system connected to an output shaft of a motor, the torque transmission structure includes a second transmission system, and the first transmission system and the second transmission system are connected; and a braking structure, wherein the braking structure is connected to the second transmission system, and the braking structure is configured to brake the second transmission system when a working state of the motor conforms to a preset condition.

In one of the embodiments, the motor further includes a brake set inside; and when the motor is energized, the brake releases the output shaft of the motor; when the motor is de-energized, the brake restricts the output shaft of the motor.

In one of the embodiments, the preset condition is that when the motor is de-energized, the brake inside the motor fails to restrict the braking of the output shaft of the motor.

In one of the embodiments, the first transmission system includes one of or at least two of a shafting transmission, a gear transmission, or a belt transmission; or the second transmission system includes one of or both of the shafting transmission and the gear transmission.

In one of the embodiments, both the first transmission system and the second transmission system include a shafting transmission, respectively; and a shafting (Y) of the first transmission system and a shafting (X) of the second transmission system are perpendicular to each other.

In one of the embodiments, the first transmission system includes a worm gear reducer.

In one of the embodiments, the second transmission system includes a reference rotary shaft and an adapter disc coaxially fixed with the reference rotary shaft, and the reference rotary shaft is connected with the first transmission system.

In one of the embodiments, the braking structure includes a de-energized brake cooperating with the reference rotary shaft; or the de-energized brake is an electromagnetic de-energized brake.

In one of the embodiments, the de-energized brake and the motor use the same power cable.

In one of the embodiments, the first transmission system is a gear transmission; a gear is coaxially fixed on the reference rotary shaft of the second transmission system; another gear is fixed on the output shaft of the motor; and the gear and the another gear are meshed successively by multiple gears.

In one of the embodiments, the first transmission system is a belt transmission; and two ends of the belt transmission are respectively connected to the output shaft of the motor and the second transmission system.

In one of the embodiments, a worm of the worm gear reducer is connected to the output shaft of the motor; and a worm wheel of the worm gear reducer is connected to the second transmission system.

In one of the embodiments, the braking structure is provided to restrict rotation of the reference rotary shaft and keep the reference rotary shaft stationary to brake the second transmission system.

In one of the embodiments, the second transmission system is a slope reducer or a rotary vector reducer in conjunction with the worm gear reducer.

A medical imaging equipment, including: a rotary driving module; an installation module, wherein the installation module is fixed in a predetermined position and the installation module has a support arm; and a C-arm being rotatably connected to the support arm and connected to the second transmission system.

In one of the embodiments, the medical imaging equipment further includes a rolling bearing; an outer ring of the rolling bearing is fixedly connected to the support arm; and an inner ring of the rolling bearing is respectively fixedly connected to the C-arm and the second transmission system.

In one of the embodiments, the medical imaging equipment further includes a rotary locking assembly; one side of the rotary locking assembly is fixedly connected to the rotary driving module; and the other side of the rotary locking assembly is fixedly connected to the support arm.

In one of the embodiments, the motor, the first transmission system, and the braking structure are assembled together to form a first assembly; the second transmission system and the rotary locking assembly are assembled together to form a second assembly; and the first assembly and the second assembly are connected by a coupling.

In one of the embodiments, the rotary locking assembly includes a support frame; and the support frame is movably mounted on a shaft of the second transmission system.

In one of the embodiments, a direction of the shafting (X) of the second transmission system is parallel to a direction of a symmetrical axis (A) of the C-arm.

In one of the embodiments, the second transmission system includes a reference rotary shaft and an adapter disc coaxially fixed with the reference rotary shaft; the reference rotary shaft is coaxially fixed with the worm wheel of the first transmission system; and the adapter disc extends outward radially along the reference rotary shaft in a disc shape, and the adapter disc is connected to the C-arm.

In one of the embodiments, the first assembly or the second assembly are detachably connected to the coupling.

In summary, in the rotary driving module and medical imaging equipment provided in this application, the rotary driving module includes a motor, a torque transmission structure, and a braking structure. The torque transmission structure includes a first transmission system and a second transmission system. The first transmission system is connected to an output shaft of the motor, and the first transmission system is connected to the second transmission system. The braking structure is connected to the second transmission system and is configured to brake the transmission power torque of the second transmission system when the working state of the motor conforms to the preset condition.

Configured in such a way, when the working condition of the motor meets the preset conditions, for example, when the braking restrict of the motor brake on the motor output shaft fails, due to the setting of the braking structure, the braking structure brakes the second transmission system, ensuring that the entire rotary driving module is not reverse-driven by the C-arm, and the C-arm remains relatively stationary after the second transmission system is braked, thereby avoiding the safety hazard caused by the loss of control of the C-arm.

It should be noted that the medical imaging equipment of the application includes the rotary driving module and thus has the beneficial technical effect brought by the rotary driving module, which may not be repeated here.

### BRIEF DESCRIPTION OF THE DRAWINGS

It should be understood by those skilled in the art that the drawings provided are intended for a better understanding of the application and do not constitute any limitation to the scope of the application. Wherein:

FIG. 1 is a schematic diagram of a medical imaging equipment of one embodiment of the present application;

FIG. 2 is a schematic diagram of an assembled rotary driving module, support arm, and C-arm of one embodiment of the present application;

FIG. 3 is a schematic diagram of an assembled rotary driving module and C-arm of one embodiment of the present application;

FIG. 4 is a schematic diagram of a rotary driving module of one embodiment of the present application;

FIG. 5 is an explosion diagram of a rotary driving module of one embodiment of the present application;

FIG. 6 is a side view of the rotary driving module in FIG. 4; and

FIG. 7 is another schematic diagram of a rotary driving module of one embodiment of the present application.

In the attached figure:
10—motor; 21—first transmission system; 22—second transmission system; 221—reference rotary shaft; 222—adapter disc; 30—braking structure; 40—C-arm; 50—installation module; 500—support arm; 60—rolling bearing; 61—inner ring; 62—outer ring; 70—rotary locking assembly; 71—support frame; 72—stoppers; 80—couplings; 100—radiation source; 110—detector.

### DETAILED DESCRIPTION

To make the purpose, advantages and features of the application clearer, further details of the application are provided below with reference to the drawings and specific embodiments. It should be noted that the drawings are in a very simplified form and are not drawn to scale, and are only intended to facilitate and clearly illustrate the purpose of the embodiments of the application. Furthermore, the structures shown in the drawings are often part of the actual structures. In particular, each drawing needs to show different focuses and sometimes uses different scales.

As used in the present application, the singular forms “one”, “one” and “that” include plural objects, the term “or” is usually used with the meaning of including “and/or”, and the term “several” is usually used with the meaning of including “at least one”, The term “at least two” is usually used to include the meaning of “two or more”, and in addition, the terms “first”, “second”, “third” are used for descriptive purposes only and should not be understood as indicating or suggesting relative importance or implicitly indicating the number of technical features indicated. Thus, a feature that is defined as “first”, “second”, “third” may explicitly or implicitly include one or at least two of those features, and “one end” and “the other end” and “the near end” and “the far end” usually refer to the two corresponding parts, which not only include endpoints, but the terms “installation”, “connection”, “connection” should be understood in a broad sense, for example, It can be a fixed connection, or a detachable connection, or as a whole; It can be a mechanical connection or an electrical connection; It can be a direct connection or an indirect connection through an intermediate medium, it can be an internal connection between two components or an interaction relationship between two components. Furthermore, as used in the present application, one element placed on another element usually only indicates a connection, coupling, fit or transmission relationship between the two elements, and the two elements can be directly or indirectly connected, coupled, fit or transmitted through an intermediate element, and cannot be understood as indicating or suggesting a spatial position relationship between the two elements, That is, one component may be located in any position within, outside, above, below or on one side of the other component, unless otherwise expressly stated in the content. For those skilled in the art, the specific meanings of the above terms in the present application can be understood on a specific basis.

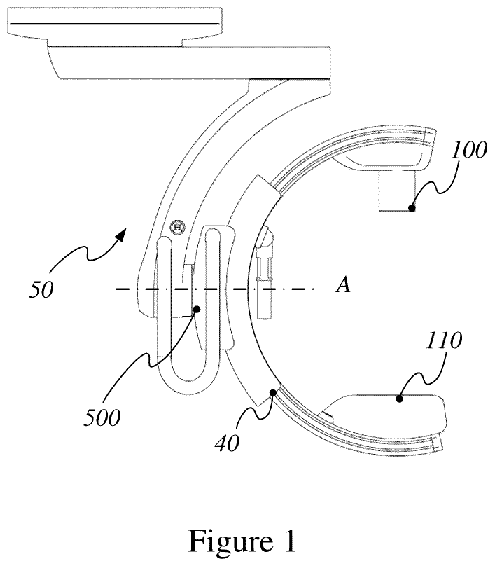

FIG. 1 is a schematic diagram of a medical imaging equipment of one embodiment of the present application. As shown in FIG. 1, this embodiment graphically presents a medical imaging equipment, and the medical imaging equipment includes an installation module 50, a C-arm 40, a radiation source 100, and a detector 110. The C-arm 40 has opposite ends, the radiation source 100 is installed on one end of the C-arm 40, the detector 110 is installed on the other end of the C-arm 40, and the radiation source 100 and the detector 110 are relatively aligned. It is understandable that the radiation source 100 is a device capable of emitting X-rays, gamma rays, or electron rays, and the detector 110 is a device capable of receiving the rays emitted by the radiation source 100. The combination of the radiation source 100 and the detector 110 enables medical examination or treatment operations. In one embodiment, the radiation source 100 includes a tube, the tube is capable of emitting rays (such as X-rays), and the detector 110 can be a flat plate detector, the rays emitted by the tube are received by the detector 110 after passing through the patient for imaging processing. The installation module 50 has a support arm 500. The support arm 500 is connected to the C-arm 40, specifically to the outer arc of the middle part of the C-arm 40. The installation module 50 can be fixed in a predetermined position, thereby fixing the C-arm 40 in a certain spatial range. The predetermined position can be, for example, on the ceiling, floor or wall. A person skilled in the art may fix the installation module 50 in the appropriate position according to the actual situation, and the fixing method may be screw fixing, adsorption fixing, etc.

To ensure sufficient flexibility of the C-arm 40, the C-arm 40 is usually rotatably connected to the support arm 500. For example, the C-arm 40 is rotatably connected to the support arm 500 around its symmetrical axis A (as shown in FIG. 1), thereby meeting the different positioning requirements between the radiation source 100 and the detector 110, and thus meeting the examination requirements of different patients. For example, the plane on which the C-arm 40 is located is parallel to the horizontal plane or perpendicular to the horizontal plane. Therefore, an embodiment of the present application also provides a rotary driving module applied to the aforementioned medical imaging equipment. The rotary driving module is connected to the C-arm 40 and is used to drive the C-arm 40 to rotate.

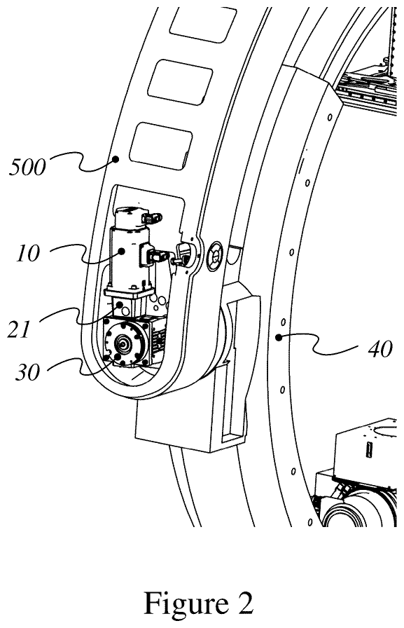

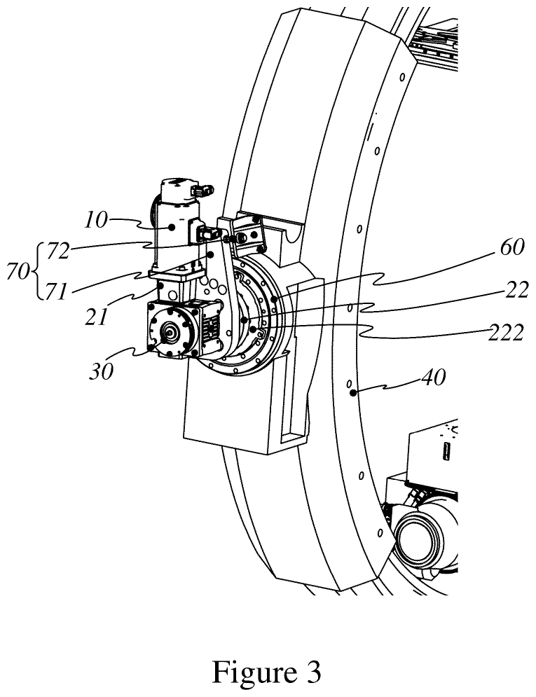

FIG. 2 is a schematic diagram of an assembled rotary driving module, the support arm 500, and the C-arm 40 of an embodiment of the present application, and FIG. 3 is a schematic diagram of an assembled rotary driving module and the C-arm 40 of one embodiment of the present application. See FIGS. 2 and 3, the rotary driving module of the embodiment of the present application includes a motor 10, a torque transmission structure, and a braking structure 30. The torque transmission structure includes a first transmission system 21 and a second transmission system 22 in coordination with the transmission of the first transmission system 21. The first transmission system 21 is connected to the output shaft of the motor 10, and the second transmission system 22 is connected to the C-arm 40, so that the power torque of the motor 10 is transmitted successively through the first transmission system 21 and the second transmission system 22 to the C-arm 40, thereby driving the C-arm 40 to rotate. The braking structure 30 is connected to the second transmission system 22. When the working state of motor 10 conforms with a preset condition, the braking structure 30 brakes the second transmission system 22, thereby putting the second transmission system 22 in a stationary state, and the C-arm 40 also remains relatively stationary. Thus, the second transmission system 22 stops its own transmission work. The greater load exerted by the C-arm 40, the radiation source 100, and detector 110 on the C-arm 40, does not reverse the entire rotary driving module through the C-arm 40, thus avoiding the safety hazard of losing control of the C-arm 40.

In one embodiment of the present application, the motor 10 includes a brake set inside and an output shaft 101. The brake is set in such a way that, when the motor 10 is energized, the brake releases the output shaft 101 of the motor 10; when the motor 10 is de-energized, the brake restricts the output shaft 101 of the motor 10. Typically, the preset condition here is that the brake of the motor 10 fails to brake the output shaft 101 of the motor 10. According to the knowledge of the motor 10, when the motor 10 is connected to the power supply, the brake releases, allowing the output shaft 101 of motor 10 to rotate normally to output the power torque outward. When the motor 10 is disconnected from the power supply, the brake locks the output shaft 101 of the motor 10, preventing external devices driven by the motor 10 from driving the output shaft 101 in reverse. Therefore, when the braking restricts of the motor 10 for the output shaft fails, the configuration of the braking structure 30 can prevent the C-arm 40 from reversely driving the rotary driving module, and the safety hazard caused by the out-of-control of the C-arm 40 can be avoided through the setting of the two-level braking protection of the brake and braking structure 30.

In one embodiment of the present application, the first transmission system 21 includes one of or at least two of a shafting transmission, a gear transmission, or a belt transmission. The second transmission system 22 includes one of or both of the shafting transmission and the gear transmission. The shafting transmission, that is, the torque of the output shaft 101 of the motor 10 is mainly transmitted through the mating direction of the shaft. The gear transmission, that is, the torque of the output shaft 101 of the motor 10 is mainly transmitted through the meshing of multiple gears or tooth tips. It is understandable that a bevel gear fit can be used to change the direction of the power torque transmission of the motor 10, for example, by adjusting the direction of transmission from the shaft direction of the output shaft 101 of the motor 10 to a direction perpendicular to the output shaft 101. The belt transmission, that is, the power torque of the motor 10 is mainly transmitted to the second transmission system 22 through the driving belt.

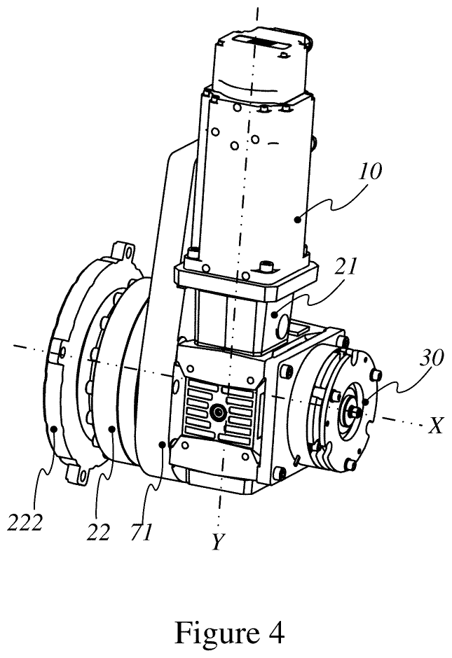

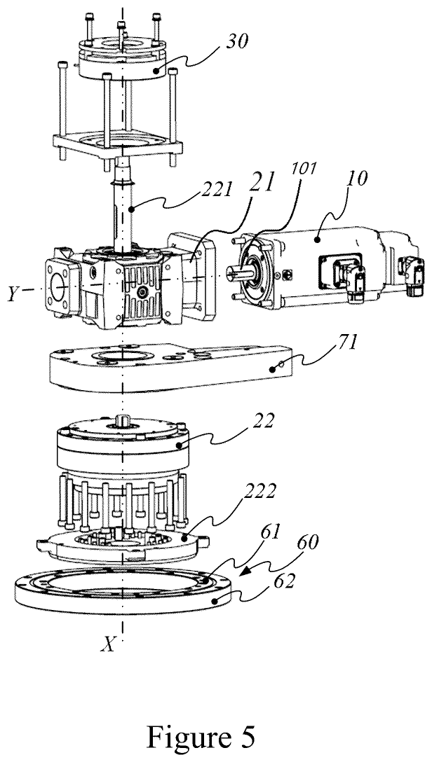

FIG. 4 is a schematic diagram of a rotating module of one embodiment of the present application. See FIG. 4, in one embodiment, the first transmission system 21 and the second transmission system 22, regardless of the number of transmission forms, both include shafting transmission, and the shafting of the first transmission system 21 and the shafting of the second transmission system 22 are perpendicular to each other. The shafting of the first transmission system 21 is denoted as shafting Y, and the shafting of the second transmission system 22 is denoted as shafting X. In some other embodiments, the first transmission system 21 may also be a belt transmission, with the two ends of the belt connected to the output shaft 101 of the motor 10 and the second transmission system 22, respectively. Or the first transmission system 21 may also be a gear transmission in which a gear is coaxially fixed on the shaft of the shafting X (i.e., the reference rotary shaft 221, FIG. 5) and another gear is fixed on the output shaft 101 of the motor 10, with the two gears meshing in sequence through several other gears. Or the first transmission system 21 is a combination of the shafting transmission and the gear transmission. It is understandable that by configuring a gear transmission system consisting of several gears, the transmission ratio can be precisely controlled to precisely control the rotational motion of the C-arm 40.

For example, for shafting Y, the first transmission system 21 includes a worm gear reducer. The worm of the worm gear reducer is connected to the output shaft 101 of the motor 10, and the worm wheel of the worm gear reducer is connected to the second transmission system 22. So that the power torque of motor 10 can be transferred from the direction of shafting Y to the direction of shafting X in coordination with the worm gear. Understandably, the direction of shafting X is parallel to the direction of the symmetrical axis A of the C-arm 40.

Illustratively, FIG. 5 is an explosion diagram of a rotary driving module of one embodiment of the present application. For shafting X, the second transmission system 22 includes a reference rotary shaft 221 and an adapter disc 222 coaxially fixed to the reference rotary shaft 221. The shaft direction of the reference rotary shaft 221 is along the direction of shafting X. The reference rotary shaft 221 is connected to the first transmission system 21. The reference rotary shaft 221 is coaxially fixed to the worm wheel of the first transmission system 21. The adapter disc 222 extends outward along the radial direction of the reference rotary shaft 221 in a disc shape, and the adapter disc 222 is connected to the C-arm 40 (as shown in FIG. 3), which increases the connection area and ensures a more stable connection between the second transmission system 22 and the C-arm 40. In this way, when the motor 10 is powered on, the brake is released, and the first transmission system 21 transmits the power torque of the motor 10 to the reference rotary shaft 221 of the second transmission system 22, driving the reference rotary shaft 221 to rotate and then the C-arm 40 to rotate under the drive of the adapter disc 222. Correspondingly, the braking structure 30 brakes the second transmission system 22 as follows: the braking structure 30 restricts the rotation of the reference rotary shaft 221, keeping the reference rotary shaft 221 stationary. The braking structure 30 relieves braking of the second transmission system 22 as follows: the braking structure 30 relieves the restriction on the rotation of the reference rotary shaft 221, allowing the reference rotary shaft 221 to rotate normally to transmit torque, or the reference rotary shaft 221 can rotate under the reverse driving of the C-arm 40. In some other embodiments, the second transmission system 22 may be a bevel reducer or a rotary vector (RV) reducer in conjunction with a worm gear reducer.

In one embodiment of the present application, the braking structure 30 includes a de-energizing brake in conjunction with the reference rotary shaft 221. Based on the working principle of the de-energized brake, when the de-energized brake is de-energized, the de-energized brake locks the reference rotary shaft 221 to restrict the rotation of the reference rotary shaft 221. When the de-energized brake is energized, the de-energized brake releases the reference rotary shaft 221 to lift the restriction on the rotation of the reference rotary shaft 221. The specific structure of the de-energized brake is known to those skilled in the art based on existing technology and is not be elaborated here.

In one embodiment of the present application, the de-energized brake and the motor 10 may use the same power cable, that is, the same power cable is connected to the power supply. Thus, when the motor 10 is de-energized but the brake of motor 10 fails, the de-energized brake can respond quickly and work to lock and brake the reference rotary shaft 221. In one embodiment, the de-energized brake is an electromagnetic de-energized brake.

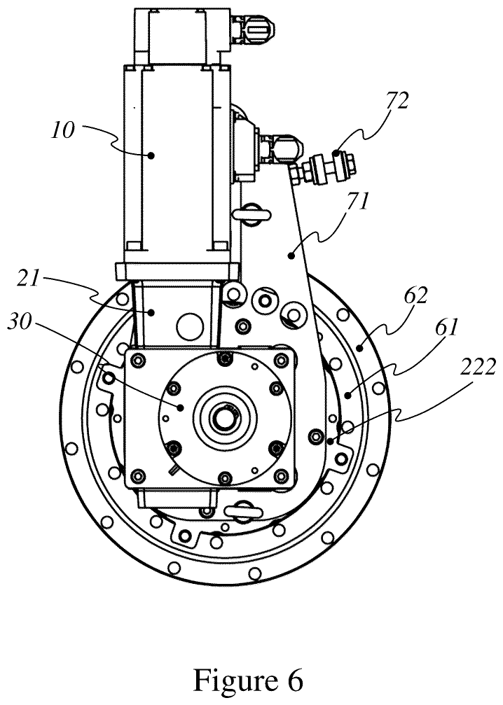

FIG. 6 is a side view of the rotary driving module in FIG. 4. See FIGS. 3, 5 and 6, in one embodiment of the present application, the medical imaging equipment also includes a rolling bearing 60. Based on the structure of the rolling bearing 60, it is known that the roller bearing 60 includes an inner ring 61, an outer ring 62, rolling elements (not illustrated) (e.g., cylindrical rollers, tapered rollers, balls, etc.), and a cage (not illustrated). The inner ring 61 is connected to an external element (e.g., the second transmission system 22, the adapter disc 222) and rotates synchronously with the external element. The outer ring 62, in conjunction with the bearing housing (in this embodiment, specifically the support arm 500), provides support. The rolling elements are evenly distributed between the inner ring 61 and the outer ring 62 by means of the cage, and shapes, sizes, and quantity of the rolling elements directly affect the performance and service life of the rolling bearing 60. The cage makes the rolling elements evenly distributed and guides the rotation of the rolling elements to play a lubricating role. The inner ring 61 can rotate coaxially relative to the outer ring 62 under the action of the rolling elements, and the coefficient of rotational friction is very small. The outer ring 62 of the rolling bearing 60 is fixedly connected to the support arm 500, for example, the outer ring 62 is fixed in the hole of the support arm 500. The inner ring 61 of the rolling bearing 60 is fixedly connected to the second transmission system 22, thus rotating after the second transmission system 22 receives the power torque of the motor 10. Specifically, the second transmission system 22 can be coaxially and detachably connected to the inner ring 61 through the adapter disc 222, and the inner ring 61 is also fixedly connected to the C-arm 40. So that the rotation of the inner ring 61 can drive the C-arm 40 to rotate, and the C-arm 40 can be considered to be loaded onto the support arm 500 through the rolling bearing 60. The setting of the rolling bearing 60 supports the second transmission system 22 and reduces the coefficient of friction during the movement of the second transmission system 22.

In one embodiment of the present application, as shown in FIG. 3, the medical imaging equipment further includes a rotary locking assembly 70, with one side of the rotary locking assembly 70 fixedly connected to the rotary driving module and the other side fixedly connected to the support arm 500, thereby locking the degrees of freedom of the rotary driving module relative to the support arm 500, Ensuring that the external structure of the rotary driving module is locked and restricted relative to the rotational degrees of freedom of the support arm 500 around the shafting X, acting as a fixed installation, and allowing the torque transmission structure inside the rotary driving module to transfer the power torque of the motor 10 to the C-arm 40.

Specifically, the rotary driving module also includes a housing (not illustrated). The torque transmission structure is located in the housing, and the torque transmission structure is movable within the housing. For example, when both the first transmission system 21 and the second transmission system 22 include shaft transmission, their respective shafts can rotate within the housing to transmit the power torque of motor 10. The rotary locking assembly 70 includes a support frame 71 and a stopper 72 set on the support frame 71 (shown in FIG. 6). The support frame 71 is fixedly connected to the housing of the rotary driving module, and the stopper 72 is in contact with the support arm 500, thus locking the rotation of the housing of the rotary driving module relative to the rotation of the support arm around the shafting X in the stopper fit of the stopper 72 and the support arm 500, ensuring that the torque transmission structure can work to transfer the power torque of the motor 10 to the C-arm.

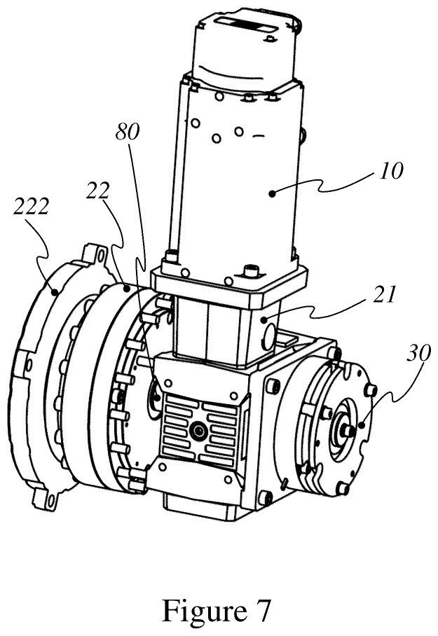

In one embodiment of the present application, both the first transmission system 21 and the second transmission system 22 include shafting transmission. The first transmission system 21 transmits the power torque to the second transmission system 22 through an internal shaft. In the case of a worm gear reducer, for example, the worm gear reducer may be configured with a shaft that is coaxially fixed to the worm wheel and the shaft is detachably connected to the reference rotary shaft 221, for example, through a coupling 80, as shown in FIG. 7, FIG. 7 is another schematic diagram of a rotary driving module of one embodiment of the present application. In this embodiment, the motor 10, the first transmission system 21, and the braking structure 30 are assembled together to form a first assembly. The second transmission system 22 and the rotary locking assembly 70 (specifically the support frame 71 of the rotary locking assembly 70, the support frame 71 is movably mounted on the shaft of the second transmission system 22, that is, movably mounted on the reference rotary shaft 221) are assembled together to form a second assembly, and the first assembly and the second assembly are connected by a coupling 80. Specifically, the first assembly and/or the second assembly are detachably connected to the coupling (80). The setting of the coupling 80 can both transmit torque and enable quick disassembly. In contrast to the problem that the structure that drives the C-arm 40 to rotate is too heavy and bulky to be disassembled and maintained in the existing technology, in this embodiment, by assembly various components, the rotary driving module can be divided into the first assembly and the second assembly. The second assembly can be installed on the C-arm 40 first by connecting the inner ring 61 of the rolling bearing 60 through the adapter disc 222, and then the second assembly can be installed on the first assembly through the coupling 80. When disassembling for maintenance, the first assembly can be removed separately for maintenance, or the first assembly can be removed first and then the second assembly. This reduces the difficulty of disassembling and maintaining the rotary drive module and enhances the serviceability of the equipment.

To sum up, in the rotary driving module and medical imaging equipment provided in the present application, the rotary driving module includes a torque transmission structure and a braking structure 30. The torque transmission structure includes a first transmission system 21 for connecting the motor 10 and a second transmission system 22 for connecting the C-arm 40. The first transmission system 21 and the second transmission system 22 are connected so that the torque transmission structure transmits the power torque of the motor 10 to the C-arm 40 in sequence through the first transmission system 21 and the second transmission system 22. The braking structure 30 is configured to brake the second transmission system 22 when the working condition of the motor 10 meets the preset condition. In such a configuration, when the motor 10 works under the preset condition, for example, when the brake of the motor 10 fails to brake the output shaft 101 of the motor 10, due to the setting of the braking structure 30, the braking structure 30 brakes the second transmission system 22, ensuring that the entire rotary driving module is not reverse-driven by the C-arm. When the second transmission system 22 is braked, it also keeps the C-arm 40 relatively stationary, avoiding the safety hazard caused by the loss of control of the C-arm 40. The medical imaging equipment of the present application includes digital subtraction angiography (DSA) equipment, which can be used to assist in the fields of angiography, cardiology, and/or neurology. The present application does not limit this. The rotary driving module of the present application is not limited to the angiographic imaging equipment, but can also be applied to all other devices with C-arm drives.

The above description is only a description of the preferred embodiment of the present application and is not any limitation of the scope of the present application. Any changes or modifications made by ordinary technicians in the art of the present application based on the above disclosure are within the protection of the technical solution of the present application.

What is claimed is:

1. A rotary driving module, including:
a torque transmission structure, wherein the torque transmission structure includes a first transmission system connected to an output shaft of a motor, the torque transmission structure includes a second transmission system, and the first transmission system and the second transmission system are connected; and
a braking structure, wherein the braking structure is connected to the second transmission system, and the braking structure is configured to brake the second transmission system when a working state of the motor conforms to a preset condition.

2. The rotary driving module of claim 1, wherein:
the motor further includes a brake set inside; and
when the motor is energized, the brake releases the output shaft of the motor; when the motor is de-energized, the brake restricts the output shaft of the motor.

3. The rotary driving module of claim 1 or 2, wherein:
the preset condition is that when the motor is de-energized, the brake inside the motor fails to restrict the braking of the output shaft of the motor.

4. The rotary driving module of any one of claims 1-3, wherein:
the first transmission system includes one of or at least two of a shafting transmission, a gear transmission, or a belt transmission; or
the second transmission system includes one of or both of the shafting transmission and the gear transmission.

5. The rotary driving module of any one of claims 1-4, wherein:
both the first transmission system and the second transmission system include a shafting transmission, respectively; and
a shafting (Y) of the first transmission system and a shafting (X) of the second transmission system are perpendicular to each other.

6. The rotary driving module of any one of claims 1-5, wherein:
the first transmission system includes a worm gear reducer.

7. The rotary driving module of any one of claims 1-6, wherein:
the second transmission system includes a reference rotary shaft and an adapter disc coaxially fixed with the reference rotary shaft, and
the reference rotary shaft is connected with the first transmission system.

8. The rotary driving module of claim 7, wherein:
the braking structure includes a de-energized brake cooperating with the reference rotary shaft; or
the de-energized brake is an electromagnetic de-energized brake.

9. The rotary driving module of claim 8, wherein:
the de-energized brake and the motor use the same power cable.

10. The rotary driving module of claim 7, wherein:
the first transmission system is a gear transmission;
a gear is coaxially fixed on the reference rotary shaft of the second transmission system;
another gear is fixed on the output shaft of the motor; and
the gear and the another gear are meshed successively by multiple gears.

11. The rotary driving module of any one of claims 1-4, wherein:
the first transmission system is a belt transmission; and
two ends of the belt transmission are respectively connected to the output shaft of the motor and the second transmission system.

12. The rotary driving module of claim 6, wherein:
a worm of the worm gear reducer is connected to the output shaft of the motor; and
a worm wheel of the worm gear reducer is connected to the second transmission system.

13. The rotary driving module of any one of claims 7-10, wherein:
the braking structure is provided to restrict rotation of the reference rotary shaft and keep the reference rotary shaft stationary to brake the second transmission system.

14. The rotary driving module of claim 6 or 12, wherein:
the second transmission system is a slope reducer or a rotary vector reducer in conjunction with the worm gear reducer.

15. A medical imaging equipment, including:
the rotary driving module of any one of claims 1-14;
an installation module, wherein the installation module is fixed in a predetermined position and the installation module has a support arm; and
a C-arm being rotatably connected to the support arm and connected to the second transmission system.

16. The medical imaging equipment of claim 15, wherein:
the medical imaging equipment further includes a rolling bearing;
an outer ring of the rolling bearing is fixedly connected to the support arm; and
an inner ring of the rolling bearing is respectively fixedly connected to the C-arm and the second transmission system.

17. The medical imaging equipment of claim 15 or 16, wherein:
the medical imaging equipment further includes a rotary locking assembly;
one side of the rotary locking assembly is fixedly connected to the rotary driving module; and
the other side of the rotary locking assembly is fixedly connected to the support arm.

18. The medical imaging equipment of claim 17, wherein:
the motor, the first transmission system, and the braking structure are assembled together to form a first assembly;
the second transmission system and the rotary locking assembly are assembled together to form a second assembly; and
the first assembly and the second assembly are connected by a coupling.

19. The medical imaging equipment of claim 17, wherein:
the rotary locking assembly includes a support frame; and
the support frame is movably mounted on a shaft of the second transmission system.

20. The medical imaging equipment of any one of claims 15-19, wherein:
a direction of the shafting (X) of the second transmission system is parallel to a direction of a symmetrical axis (A) of the C-arm.

21. The medical imaging of any one of claims 15-20, wherein:
the second transmission system includes a reference rotary shaft and an adapter disc coaxially fixed with the reference rotary shaft;
the reference rotary shaft is coaxially fixed with the worm wheel of the first transmission system; and
the adapter disc extends outward radially along the reference rotary shaft in a disc shape, and the adapter disc is connected to the C-arm.

22. The medical imaging equipment of claim 18, wherein:
the first assembly or the second assembly are detachably connected to the coupling.
